(19) United States
(12) Patent Application Publication
(10) Pub. No.: US 2025/0204927 A1
(43) Pub. Date: Jun. 26, 2025

(54) SHAPED SHAPE MEMORY FOAM IMPLANTS FOR OCCLUDING LEFT ATRIAL APPENDAGE

(71) Applicant: BOSTON SCIENTIFIC SCIMED, INC,, MAPLE GROVE, MN (US)

(72) Inventors: Patrick Willoughby, Shoreview, MN (US); Scott Andrew McCullough, Plymouth, MN (US)

(73) Assignee: BOSTON SCIENTIFIC SCIMED, INC., MAPLE GROVE, MN (US)

(21) Appl. No.: 18/987,200

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00

(52) U.S. Cl. CPC A61B 17/1219; A61B 17/12031; A61B 17/12122; A61B 2017/00526; A61B 2017/00867

(57) ABSTRACT

An occlusive device may include a shape memory foam component having a compressed configuration in which the shape memory foam component has a first shape and an expanded configuration in which the shape memory foam component has a second shape that is different from the first shape. The occlusive device may be adapted to occlude a left atrial appendage (LAA), for example. The shape memory foam component may be formed by crimping a shape memory foam blank into an inverse shape that is an inverse of the second shape and subsequently cutting the shape memory foam component from the crimped shape memory foam blank, the shape memory foam component having the first shape.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for occluding a left atrial appendage.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding the left atrial appendage (LAA). The LAAC device includes a shape memory foam component having a compressed configuration in which the shape memory foam component has a first shape that is adapted for delivery and an expanded configuration in which the shape memory foam component has a second shape that different from the first shape and is adapted to occlude a left atrial appendage (LAA). The shape memory foam component is formed by crimping a shape memory foam blank into an inverse shape that is an inverse of the second shape, and subsequently cutting the shape memory foam component from the crimped shape memory foam blank, the shape memory foam component having the first shape.

Alternatively or additionally, the second shape may include a complex shape.

Alternatively or additionally, the first shape may include a cylindrical shape and the second shape may include a non-cylindrical shape.

Alternatively or additionally, the second shape may be adapted to occlude an LAA having a profile that resembles a chicken wing.

Alternatively or additionally, the second shape may be adapted to occlude an LAA having a profile that resembles a windsock.

Alternatively or additionally, the second shape may be adapted to occlude an LAA having a profile that resembles a cactus.

Alternatively or additionally, the second shape may be adapted to occlude an LAA having a profile that resembles a cauliflower.

Alternatively or additionally, the second shape may be adapted to occlude an LAA having a double lobe.

Alternatively or additionally, the shape memory foam component may be adapted to change from the first shape to the second shape when exposed to increased temperature and/or increased moisture.

Another example may be found in a method for manufacturing an occlusive device that includes a shape memory foam component having a compressed configuration in which the shape memory foam component has a first shape and an expanded configuration in which the shape memory foam component has a second shape different from the first shape. The method includes crimping a shape memory foam blank into an inverse shape that is an inverse of the second shape, heat setting the crimped shape memory foam blank to temporarily hold the crimped shape memory foam blank in the inverse shape, and subsequently cutting the shape memory foam component from the crimped shape memory foam blank. The shape memory foam component has the first shape.

Alternatively or additionally, the method may further include an initial step of cutting the shape memory foam blank out of a larger piece of shape memory foam prior to crimping the shape memory foam blank.

Alternatively or additionally, the shape memory foam component may be adapted to change from the first shape to the second shape when exposed to increased temperature and/or increased moisture.

Alternatively or additionally, the first shape may include a cylindrical shape.

Alternatively or additionally, the second shape may include a non-cylindrical shape.

Alternatively or additionally, the second shape may be adapted to occlude a left atrial appendage (LAA).

Alternatively or additionally, the second shape may be adapted to occlude a passage within a previously implanted device.

Another example may be found in a method for manufacturing a left atrial appendage closure (LAAC) device that includes a shape memory foam component having a compressed configuration in which the shape memory foam component has a first shape that is adapted for delivery and an expanded configuration in which the shape memory foam component has a second shape that different from the first shape and is adapted to occlude a left atrial appendage (LAA). The method includes placing a shape memory foam blank into a mold that is adapted to crimp the shape memory foam blank into an inverse shape that is an inverse of the expanded configuration and using the mold to crimp the shape memory foam blank. The crimped shape memory foam blank is heat set to temporarily hold the crimped shape memory foam blank in the inverse shape. The shape memory foam component is cut from the crimped shape memory foam blank. The shape memory foam component has the compressed configuration.

Alternatively or additionally, the shape memory foam component may be adapted to change from the first shape to the second shape when exposed to increased temperature and/or increased moisture.

Alternatively or additionally, the first shape may include a cylindrical shape.

Alternatively or additionally, the second shape may include a non-cylindrical shape.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIGS. 2A, 2B, 2C, 2D, 2E and 2F are partial cross-sectional views of particular profiles of various left atrial appendages;

FIGS. 3A, 3B, 3C, 3D, 3E and 3F are partial cross-sectional views of the various left atrial appendages with various occlusive devices disposed therein;

FIG. 4 is a schematic view of creating a shape memory foam blank;

FIG. 5 is a schematic step-by-step view of creating a shape memory foam component from the shape memory foam blank of FIG. 4, showing the shape memory foam component in its compressed configuration and in its expanded configuration;

FIG. 6 is a schematic view of a mold profile and a shape memory foam blank disposed within the mold profile;

FIG. 7 is a schematic view of a shape memory component formed from the shape memory foam blank of FIG. 6, shown in its expanded configuration;

FIG. 8 is a schematic step-by-step view of creating a shape memory foam component from a shape memory foam blank, showing the shape memory foam component in its compressed configuration and in its expanded configuration;

FIG. 9 is a schematic view showing use of the shape memory foam component formed in FIG. 8; and

FIG. 10 is a schematic view showing a possible use of a shape memory foam component, with the shape memory foam component shown in its compressed configuration and its expanded configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary clement shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Shape memory foam components may be used in a variety of applications. As an illustrative but non-limiting example, shape memory foam components may be used in forming a left atrial appendage closure (LAAC) device that is adapted for occluding a left atrial appendage. An example left atrial appendage closure (LAAC) device that is adapted for occluding the left atrial appendage (LAA) includes a shape memory foam component. The shape memory foam component has a compressed configuration in which the shape memory foam component has a first shape that is adapted for delivery and an expanded configuration in which the shape memory foam component has a second shape that is different from the first shape and is adapted to occlude a left atrial appendage (LAA). The shape memory foam component is formed via a process that includes crimping a shape memory foam blank into an inverse shape that is an inverse of a second shape and subsequently cutting the shape memory foam component from the crimped shape memory foam blank, the shape memory foam component having the first shape. The second shape may include a complex shape such as a non-cylindrical shape while the first shape may include a cylindrical shape. The second shape may be adapted to occlude an LAA having a profile that resembles a chicken wing. The second shape may be adapted to occlude an LAA having a profile that resembles a windsock. The second shape may be adapted to occlude an LAA having a profile that resembles a cactus. The second shape may be adapted to occlude an LAA having a profile that resembles a cauliflower. The second shape may be adapted to occlude an LAA having two lobes. The shape memory foam component may be adapted to change from the first shape to the second shape when exposed to increased temperature and/or increased moisture, such as when the shape memory foam component is exposed to blood within a patient. In some cases, the shape memory foam component may change from the first shape to the second shape as a result of temperature, moisture, chemical and electrical exposure, and combinations thereof.

An occlusive device includes a shape memory foam component having a compressed configuration in which the shape memory foam component has a first shape and an expanded configuration in which the shape memory foam component has a second shape different from the first shape. A method for manufacturing the occlusive device includes crimping a shape memory foam blank into an inverse shape that is an inverse of the second shape, heat setting the crimped shape memory foam blank to temporarily hold the crimped shape memory foam blank in the inverse shape, and subsequently cutting the shape memory foam component from the crimped shape memory foam blank. The shape memory foam component cut from the crimped shape memory foam blank has the first shape. The method may further include an initial step of cutting the shape memory foam blank out of a larger piece of shape memory foam prior to crimping the shape memory foam blank. The shape memory foam component may be adapted to change from the first shape to the second shape when exposed to increased temperature and/or increased moisture. The first shape may include a cylindrical shape. The second shape may include a non-cylindrical shape. The second shape may be adapted to occlude a left atrial appendage (LAA). The second shape may be adapted to occlude a passage within a previously implanted device.

A left atrial appendage closure (LAAC) device includes a shape memory foam component having a compressed configuration in which the shape memory foam component has a first shape that is adapted for delivery and an expanded configuration in which the shape memory foam component has a second shape that different from the first shape and is adapted to occlude a left atrial appendage (LAA). A method of manufacturing the LAAC device includes placing a shape memory foam blank into a mold that is adapted to crimp the shape memory foam blank into an inverse shape that is an inverse of the expanded configuration, using the mold to crimp the shape memory foam blank, heat setting the crimped shape memory foam blank to temporarily hold the crimped shape memory foam blank in the inverse shape, and cutting the shape memory foam component from the crimped shape memory foam blank. The shape memory foam component has the compressed configuration. The shape memory foam component may be adapted to change from the first shape to the second shape when exposed to increased temperature and/or increased moisture, such as when exposed to blood. The first shape may include a cylindrical shape. The second shape may include a non-cylindrical shape.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

In some cases, a doughnut-shaped piece of shape memory foam (from which a cylindrical shape has been removed) may be deployed on the outside of various cylindrical-shaped devices to help with anchoring, to reduce leakage around the device, and so on. In some cases, when occluding left atrial appendages, an occlusive device may be custom-designed to fit a particular left atrial appendage. This may include using three dimensional imaging to determine an appropriate shape for making a custom mold for crimping and then cutting a custom-made occlusive device for a particular patient, including a three dimensional profile matching that patient's left atrial appendage. In some instances, a number of occlusive devices may be combined into a kit, where the kit may include a number of uniquely shaped (when expanded) occlusive devices that are each designed for a particular shape of left atrial appendage.

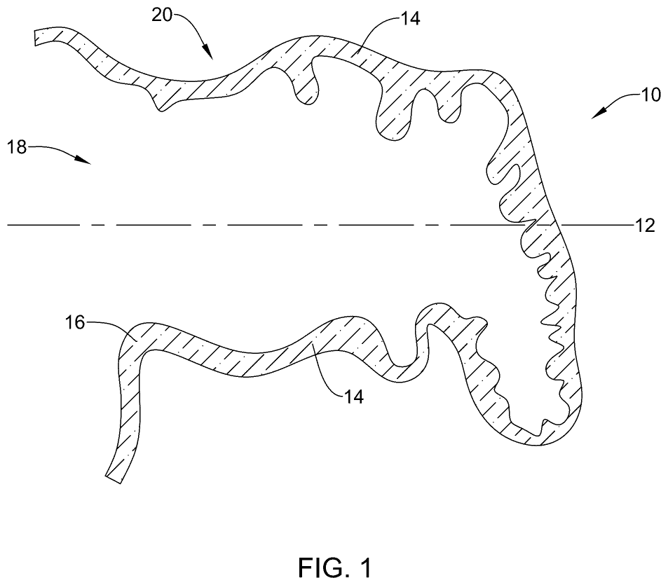

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

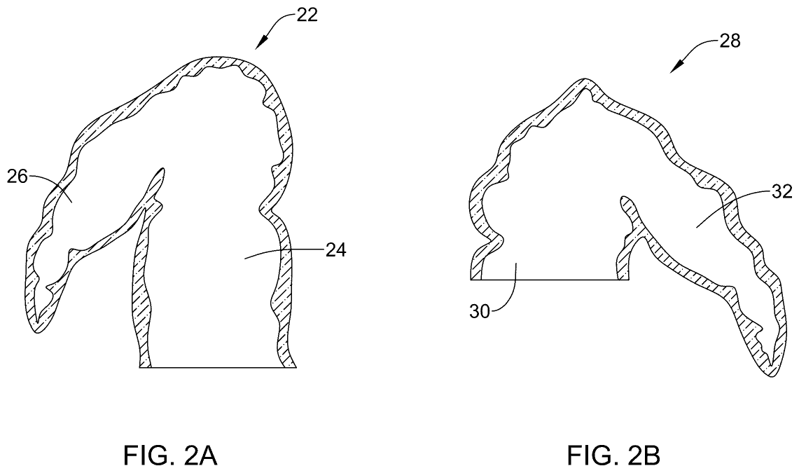

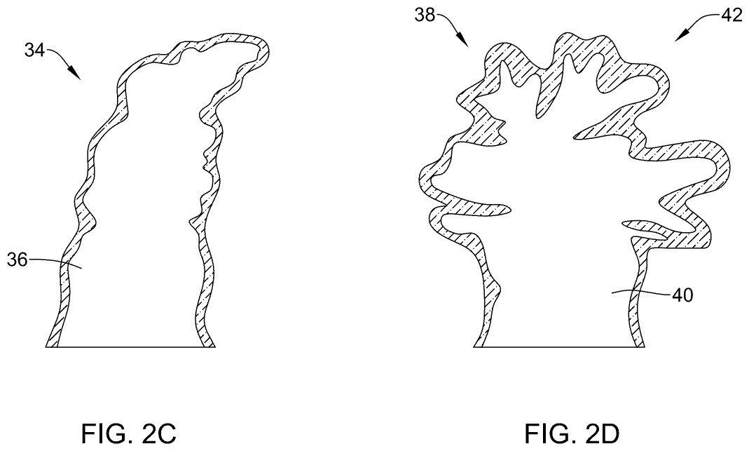

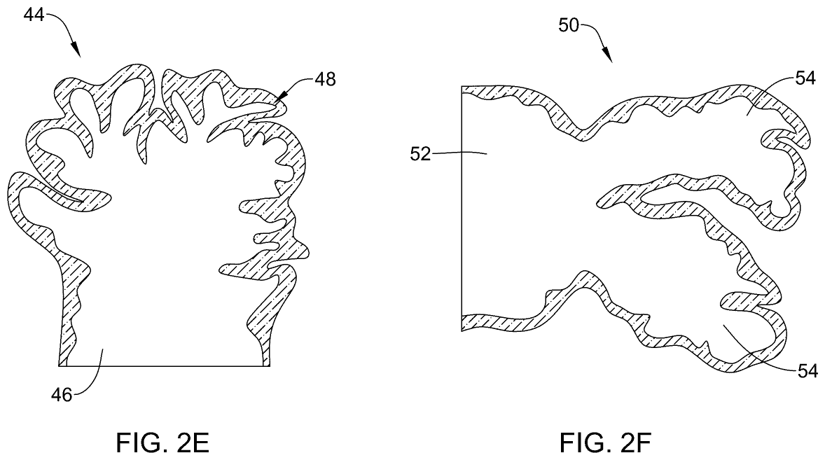

It will be appreciated that FIG. 1 shows an LAA 10 that is just one example of what a left atrial appendage may look like. While some patients may have a left atrial appendage that looks similar to the LAA 10, some patients have a left atrial appendage that may have a different shape from the LAA 10. FIGS. 2A-2F are partial cross-sectional views providing illustrative left atrial appendage shapes. It will be appreciated that this is not intended to be exhaustive, but merely illustrative, as the shape memory foam components described herein may be used in any possible left atrial appendage shape.

FIG. 2A shows an LAA 22 that is commonly referred to as having a “chicken wing” profile that includes a main segment 24 and a terminal segment 26 that may be positioned at an angle with respect to the main segment 24. A cylindrical occlusive device implanted in the LAA 22 may at least partially occlude and/or fill the main segment 24, but may not fill the terminal segment 26. FIG. 2B shows an LAA 28 that may be referred to as having a “short neck chicken wing” profile that includes a main segment 30 and a terminal segment 32 that may be positioned at an angle with respect to the main segment 30. In comparing to the LAA 22, the main segment 30 of the LAA 28 may be seen as being shorter than the main segment 24. A cylindrical occlusive device implanted in the LAA 28 may at least partially occlude and/or fill the main segment 30, but may not fill the terminal segment 32.

FIG. 2C shows an LAA 34 that is commonly referred to having a “windsock” profile. The LAA 34 includes a main segment 36 that tapers distally. A cylindrical occlusive device implanted within the LAA 34 may not fully occlude the LAA 34. FIG. 2D shows an LAA 38 that is commonly referred to as having a “cactus” profile. The LAA 38 includes a main segment 40 and a number of terminal segments 42. FIG. 2E shows an LAA 44 that is commonly referred to as having a “cauliflower” profile. The LAA 44 includes a main segment 46 and a number of terminal segments 48. FIG. 2F shows an LAA 50 that is commonly referred to as having a “double lobe” profile. The LAA 50 includes a main segment 52 and a pair of terminal segments 54.

In some cases, each of the LAA 22, the LAA 28, the LAA 34, the LAA 38, the LAA 44 and/or the LAA 50 may be occluded using two or more occlusive foam implants each having a simple cylindrical shape. However, in some cases, a single occlusive foam implant may be made having a more complex shape that better fills more of the volume within any of the LAA 22, the LAA 28, the LAA 34, the LAA 38, the LAA 44 and/or the LAA 50. In some cases, a single occlusive foam implant may be made having a complex shape that is designed to fill any left atrial appendage, including left atrial appendages having shapes that are different from those shown herein.

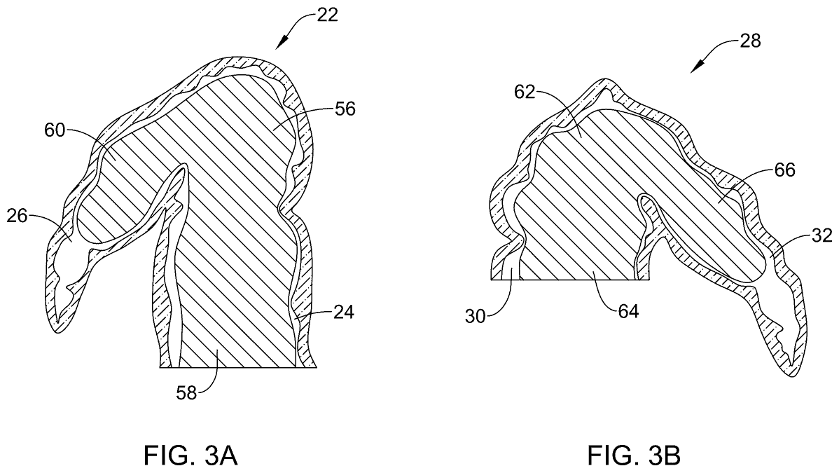

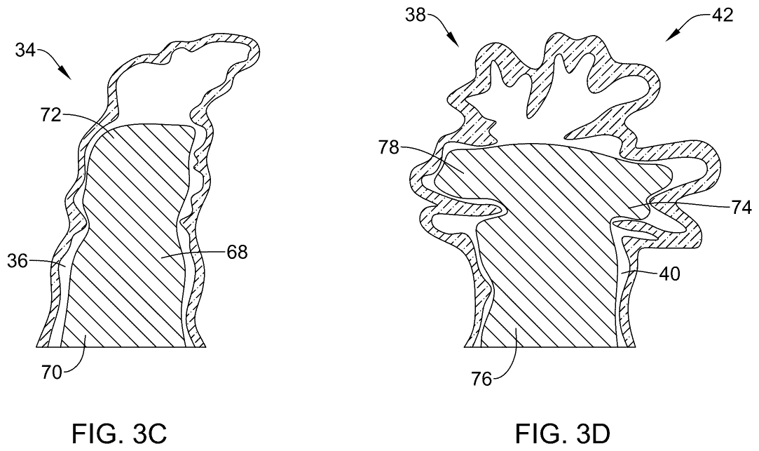

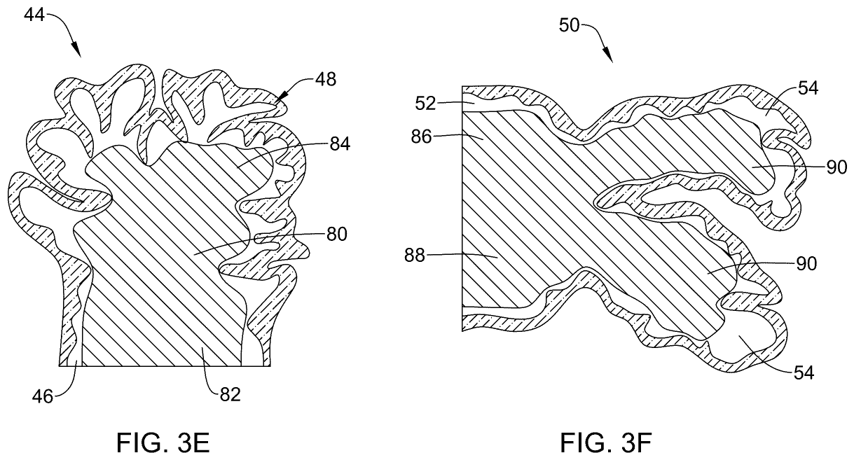

In FIG. 3A, the LAA 22 may be seen as being occluded by an occlusive foam implant 56, where the occlusive foam implant 56 includes a portion 58 that fits within the main segment 24 and a portion 60 that fits within the terminal segment 26. As an example, the occlusive foam implant 56 may be formed with the portion 58 having a cylindrical shape dimensioned to fit within the main segment 24 and the portion 60 having a cylindrical shape dimensioned to fit within the terminal segment 26. The portion 60 may be axially aligned with the portion 58 until the occlusive foam implant 56 is advanced into the LAA 22. In some cases, the occlusive foam implant 56 may be manufactured having an overall profile closer to that shown in FIG. 3A, with the portion 60 extending laterally from the portion 58.

In FIG. 3B, the LAA 28 may be seen as being occluded by an occlusive foam implant 62, where the occlusive foam implant 56 includes a portion 64 that fits within the main segment 30 and a portion 66 that fits within the terminal segment 32. As an example, the occlusive foam implant 62 may be formed with the portion 64 having a cylindrical shape dimensioned to fit within the main segment 30 and the portion 66 having a cylindrical shape dimensioned to fit within the terminal segment 32. The portion 64 may be axially aligned with the portion 66 until the occlusive foam implant 62 is advanced into the LAA 28. In some cases, the occlusive foam implant 62 may be manufactured having an overall profile closer to that shown in FIG. 3B, with the portion 66 extending laterally from the portion 64.

In FIG. 3C, the LAA 34 may be seen as being occluded by an occlusive foam implant 68 that tapers from a distal end 70 to a proximal end 72. In some instances, the occlusive foam implant 68 may be considered as having a frustoconical shape.

In FIG. 3D, the LAA 38 may be seen as being occluded by an occlusive foam implant 74, where the occlusive foam implant 74 includes a portion 76 having a cylindrical shape that is dimensioned to fit within the main segment 40 and a portion 78 having a widened cylindrical shape that is dimensioned to fit between at least some of the terminal segments 42.

In FIG. 3E, the LAA 44 may be seen as being occluded by an occlusive foam implant 80, where the occlusive foam implant 80 includes a portion 82 having a cylindrical shape that is dimensioned to fit within the main segment 46 and a portion 78 having a widened cylindrical shape that is dimensioned to fit between at least some of the terminal segments 48.

In FIG. 3F, the LAA 50 may be seen as being occluded by an occlusive foam implant 86, where the occlusive foam implant 86 includes a portion 88 having a cylindrical shape that is dimensioned to fit within the main segment 52 and a pair of portions 90 that each have a cylindrical shape that is dimensioned to fit within one of the terminal segments 54. In some cases, the occlusive foam implant 86 may be formed to include the portion 88 and one of the portions 90, and the other of the portions 90 may be separately implanted prior to implanting the occlusive foam implant 86.

A variety of different shapes, including a variety of complex shapes, may be imparted to any of the occlusive foam implants 56, 62, 68, 74, 80 and 86. Any of the occlusive foam implants 56, 62, 68, 74, 80 and 86 may be formed having a simple shape such as a cylindrical shape for delivery and implantation, and may morph into a more complex shape as a result of exposure to an increased temperature and/or increased moisture once implanted as a result of exposure to blood. Any of the occlusive foam implants 56, 62, 68, 74, 80 and 86 may be made by obtaining or creating a shape memory foam blank, and then compressing the shape memory foam blank into a shape that is an inverse of a desired shape. The inverse shape may then be cut to form a shape memory foam component that forms part of the occlusive foam implant 56, 62, 68, 74, 80 and 86, or actually is the occlusive foam implant 56, 62, 68, 74, 80 and 86.

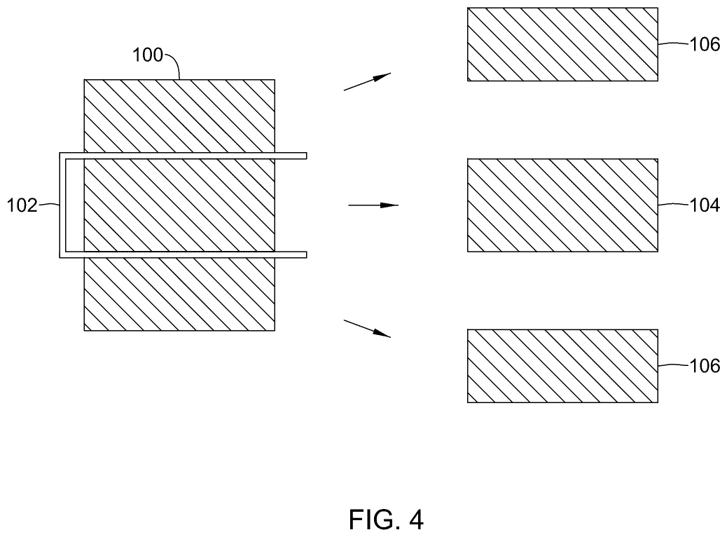

FIG. 4 provides a schematic view of forming a shape memory foam blank. A shape memory source foam 100 is provided. The shape memory source foam 100 may be produced in any desired way, using any desired polymeric precursors. As an example, the shape memory source foam 100 may be produced in a large container batch synthesis, or via a reactive extrusion process. The resulting shape memory source foam 100 may have a consistent interior, but may have a thick and closed skin along its outer surface. Accordingly, trying to form a desired complex shape using a mold will not produce a desired result. A cutting tool 102 is used to cut a shape memory foam blank 104 out of the shape memory source foam 100. While the cutting tool 102 is illustrated as simply cutting a cylinder out of the shape memory source foam 100, it will be appreciated that additional cuts may be performed in order to produce the shape memory foam blank 104 having a desired uniform texture, density and other properties. While not shown, in some cases the shape memory foam blank 104 may be cut to have a non-cylindrical shape. Several pieces 106 from the shape memory source foam 100 may be discarded, for example.

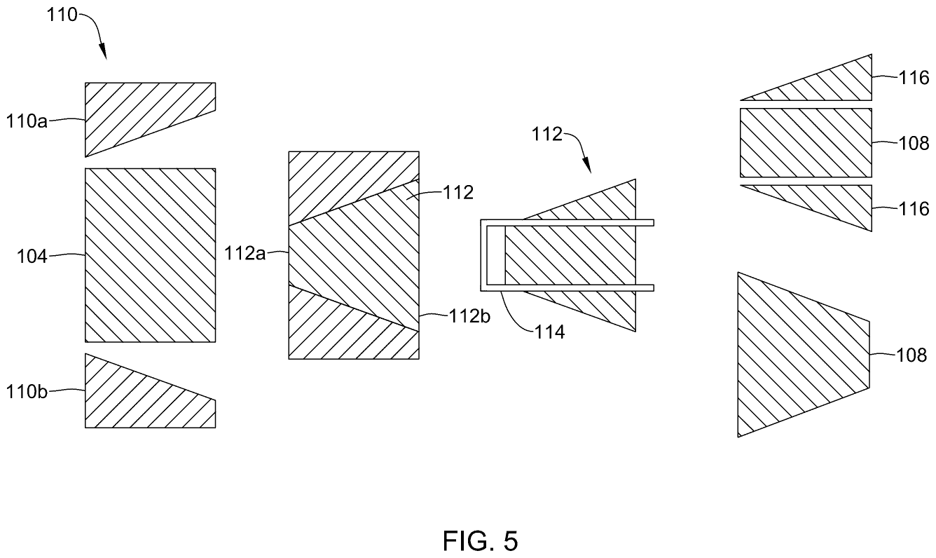

FIG. 5 provides a schematic view of a step-by-step process of converting the shape memory foam blank 104 into a desired shape memory foam component 108. On the left hand side of FIG. 5, the shape memory foam blank 104 may be loaded into a crimping device 110 that as shown, may include a first crimper 110a and a second crimper 110b. The crimping device 110 may be machined, molded or 3D printed out of any suitable material, for example. Next, the first crimper 110a and the second crimper 110b are brought closer together in order to crimp the shape memory foam blank 104 into a crimped inverse foam piece 112. In some cases, heat may be applied to the crimped inverse foam piece 112 to heat-set the crimped inverse foam piece 112 in order to help the crimped inverse foam piece 112 to retain its shape. Next, a cutting tool 114 is used to cut the shape memory foam component 108 out of the crimped inverse foam piece 112. This results in the shape memory foam component 108 in its compressed configuration, as shown on the upper right hand side of FIG. 5. The remaining pieces 116 may be discarded.

The shape memory foam component 108, which may then form part or all of an occlusive foam implant, has a compressed configuration in which the shape memory foam component 108 has a first shape. As an example, the first shape may be a cylinder, as shown in the upper right hand side of FIG. 5. Once implanted and exposed to increased temperature and increased moisture as a result of exposure to blood, the shape memory foam component 108 may move into a second shape as the shape memory foam component 108 expands. As shown, the shape memory foam component 108 has an expanded shape that is an inverse of the crimped inverse foam piece 112. This is because when the crimped inverse foam piece 112 is formed, the foam at the left hand side 112a has a greater density (as a result of compression) than the foam at the right hand side 112b. When a cylinder is cut out of the crimped inverse foam piece 112 to form the shape memory foam component 108, this difference in density remains. When the shape memory foam material expands, the foam that was more compressed will expand more and the foam that was compressed less will expand less. Any foam that was not compressed will not expand. Accordingly, this provides a way to make a shape memory foam component that is convertible between a first or compressed configuration for delivery and implantation and a second or expanded configuration subsequent to implantation. In some cases, the shape memory foam component may be made to have a simple shape in the first or compressed configuration and a complex shape in the second or expanded configuration.

When the shape memory foam component expands, significantly compressed regions will attempt to expand fully to their original size while the lightly compressed regions will expand to a lesser position. This leaves an inherent tension between the regions as a boundary between the regions will want to have a step change in expanded size. Since they are connected together, the step change cannot exist. Therefore, the significantly compressed region will try to pull the lightly compressed region out more, while the lightly compressed region will try to pull the significantly compressed region in more. The balance of those forces will result in neither region reaching its free expansion if it were cut separate from the other regions. There will be some tapering or sinusoidal type of pattern resulting from the balance of these forces as the regions try to find their stable geometric position. The exact pattern will depend on the stiffness of the foam, the amount of compression, and the length of the different regions.

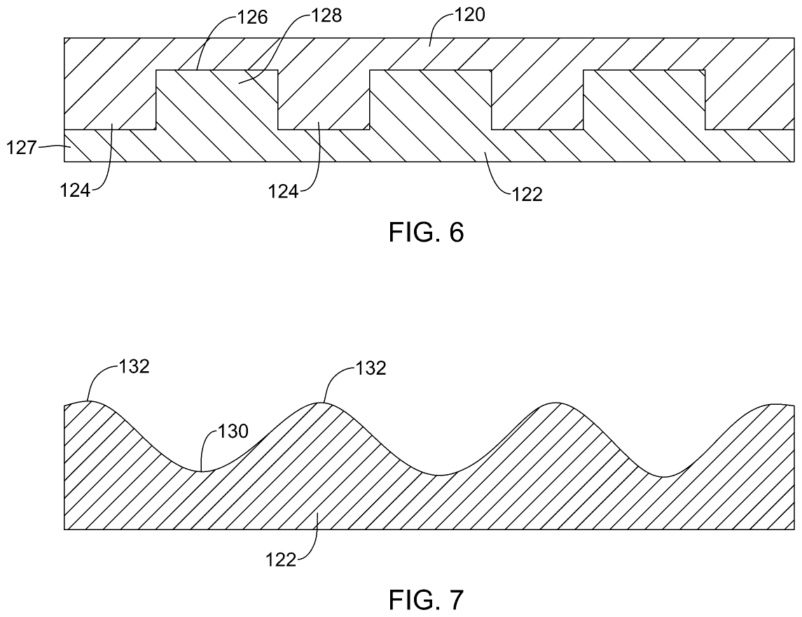

FIGS. 6 and 7 provide another example of how the shape of a crimper impacts the shape of the shape memory foam after expansion. In FIG. 6, a crimper 120 is positioned against a piece of shape memory foam 122. The crimper 120 may be machined, molded or 3D printed out of any suitable material, for example. It can be seen that the crimper 120 includes tabs 124 and intervening recesses 126, and as a result, the piece of shape memory foam 122 has corresponding regions 127 of reduced height (corresponding to where the tabs 124 are) and regions 128 of increased height (corresponding to where the recesses 126 are). The regions 127 of reduced height correspond to where the piece of shape memory foam 122 has been crimped, or compressed, while the regions 128 of increased height correspond to where the piece of shape memory foam 122 was not crimped, or compressed, or at least was crimped or compressed less.

In FIG. 7, the piece of shape memory foam 122 is shown in its expanded configuration. The piece of shape memory foam 122 includes peaks 132 that correspond to where the piece of shape memory foam 122 was compressed (or was compressed more) and intervening valleys 130 that correspond to where the piece of shape memory foam 122 was not compressed (or was compressed less). It will be appreciated that the piece of shape memory foam 122, in its expanded configuration, represents a rounded over version of the profile of the crimper 120, rather than emulating the step-wise profile of the crimper 120. The stiffness of the foam constrains the foam from following the step-wise profile of the crimper 120 as heavily crimped regions try to pull lightly crimped regions out more and lightly compressed regions will try to pull heavily crimped regions in more. As a result, the foam expands into a rounded over version of the profile of the crimper 120.

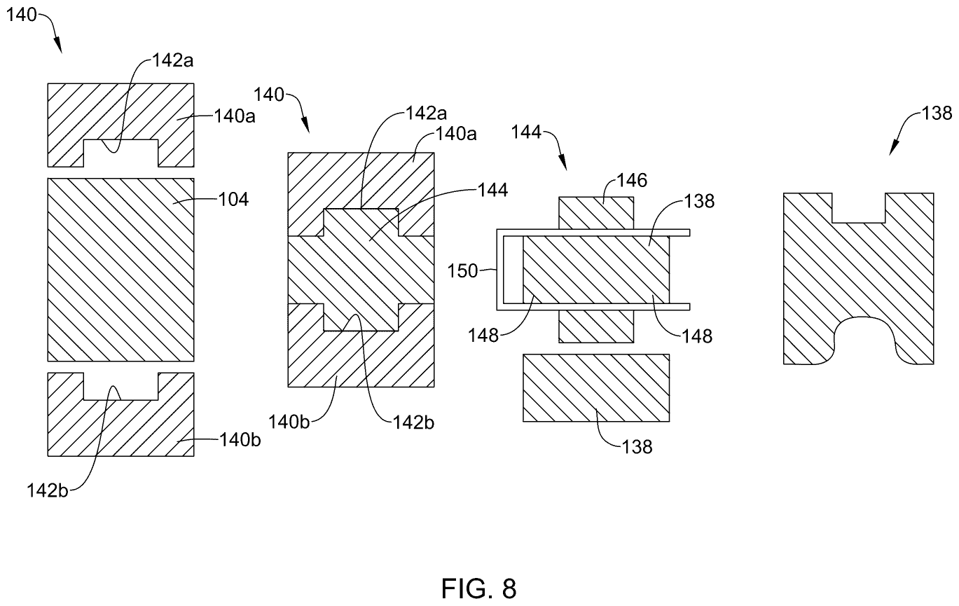

FIG. 8 provides a schematic view of a step-by-step process of converting the shape memory foam blank 104 into a desired shape memory foam component 138. On the left hand side of FIG. 8, the shape memory foam blank 104 may be loaded into a crimping device 140 that as shown, may include a first crimper 140a and a second crimper 140b. The crimping device 140 may be machined, molded or 3D printed out of any suitable material, for example. Each of the first crimper 140a and the second crimper 140b each have a profile including a recess 142a or 142b. Next, the first crimper 140a and the second crimper 140b are brought closer together in order to crimp the shape memory foam blank 104 into a crimped inverse foam piece 144. In some cases, heat may be applied to the crimped inverse foam piece 144 to heat-set the crimped inverse foam piece 144 in order to help the crimped inverse foam piece 144 to retain its shape. As can be seen, the crimped inverse foam piece 144 includes a central region 146 that is compressed less that the end regions 148. The central region 146 corresponds to where the recesses 142a and 142b are in the first crimper 140a and the second crimper 140b.

Next, a cutting tool 150 is used to cut the shape memory foam component 138 out of the crimped inverse foam piece 144. This results in the shape memory foam component 138 in its compressed configuration, as shown on the lower middle of FIG. 8. The remaining pieces may be discarded. The shape memory foam component 138, which may then form part or all of an occlusive foam implant, has a compressed configuration in which the shape memory foam component 138 has a first shape. As an example, the first shape may be a cylinder, as shown in the lower middle of FIG. 8. Once implanted and exposed to increased temperature and increased moisture as a result of exposure to blood, the shape memory foam component 138 may move into a second shape as the shape memory foam component 138 expands, as shown on the right hand side of FIG. 8. As shown, the shape memory foam component 138 has an expanded shape that is an inverse of the crimped inverse foam piece 144.

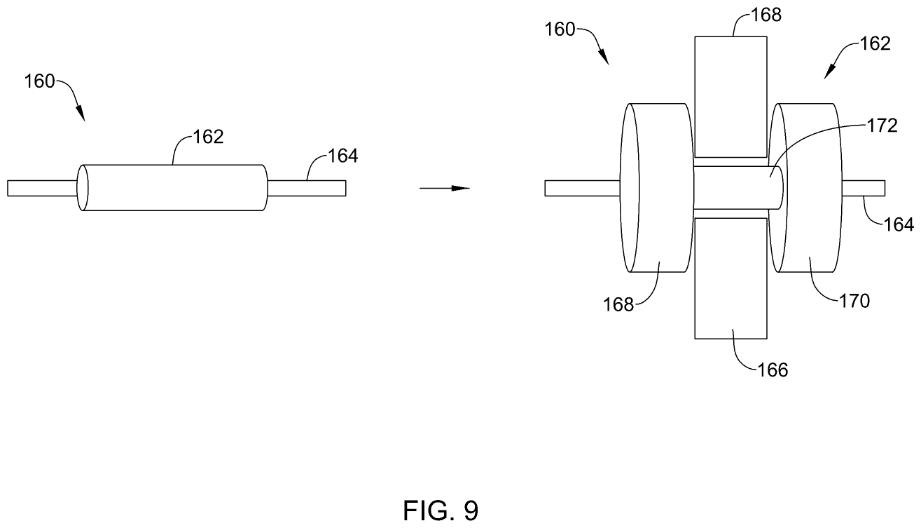

FIG. 9 provides a schematic view of an application of a shape memory foam component other than occluding a left atrial appendage such as any of the LAA 10, the LAA 22, the LAA 28, the LAA 34, the LAA 38, the LAA 44 and/or the LAA 50. On the left hand side of FIG. 9, an occlusive device 160 includes a shape memory foam component 162 that is shown in its compressed configuration, and secured to a support structure 164. The occlusive device 160 may be advanced through an opening 166 that has been formed within tissue 168. In some cases, the opening 166 may represent an opening formed within a septal wall, or an arterial or venous access site.

Upon implantation, and exposure to blood, the shape memory foam component 162 will expand into its expanded configuration, as shown on the right hand side of FIG. 9. As can be seen, in its expanded configuration, the shape memory foam component 162 includes a first disk 168 that is adapted to remain on a first side of the tissue 168 and a second disk 170 that is adapted to remain on a second side of the tissue 168, with an intervening cylindrical portion 172 extending through the opening 166 and securing the first disk 168 and the second disk 170 together. In some cases, the occlusive device 160 may effectively close off the opening 166. In some cases, the shape memory foam component 162 may be formed in a manner similar to that shown in FIG. 8.

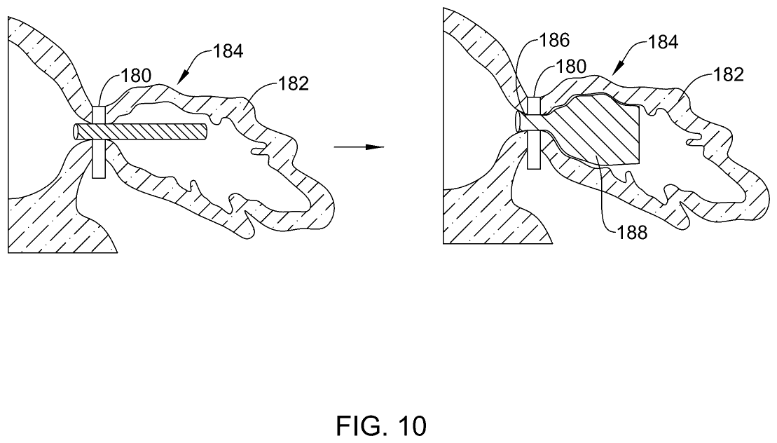

FIG. 10 is a schematic view of using an occlusive device to close an opening that remains, or extends through, an implantable medical device that has already been implanted. As shown in FIG. 10, a first occlusive device 180 has already been implanted in order to close off an LAA 182, such as during a Laminar or clip-type procedure. Other procedures including surgical procedures and catheter-based procedures are known for reducing the LAA ostium or even eliminating the LAA by running a suture around or through the LAA. An example of a catheter-based procedure is known commercially as the LARIAT™ procedure.

A second occlusive device 184 that includes or is a shape memory foam component may be extended through an opening within or formed by the first occlusive device 180, as shown on the left hand side of FIG. 10. After exposure to blood, the second occlusive device 184 will expand, as shown on the right hand side of FIG. 10. As shown, in its expanded configuration, the second occlusive device 184 may include an unexpanded portion 186 where the second occlusive device 184 extends through the opening within or formed by the first occlusive device 180, and may include an expanded portion 188 within the LAA 182 that prevents the second occlusive device 184 from moving proximally. While not shown, a proximal end of the second occlusive device 184 may also expand to help limit relative distal movement of the second occlusive device 184.

The expandable foam may include any suitable material, such as a suitable polymeric material, that is capable of transitioning from an initial configuration to an expanded configuration upon being subjected to a specific temperature or temperature range and/or exposure to moisture, such as may occur when the expandable foam is exposed to blood, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly (beta-hydroxybutyrate), polygluconate including poly-D-gluconate, poly-L-gluconate, poly-D,L-gluconate, poly (epsilon-caprolactone), poly (delta-valerolactone), poly (p-dioxanone), poly (lactide-co-glycolide) (PLGA), poly (lactide-codelta-valerolactone), poly (lactide-co-epsilon-caprolactone), poly (lactide-co-beta-malic acid), poly (beta-hydroxybutyrate-co-beta hydroxyvalerate), poly[1,3bis (p-carboxyphenoxy) propane-co-sebacic acid], and poly (sebacic acid-co-fumaric acid); (b) polycarbonate homopolymers and copolymers such as poly (trimethylene carbonate), poly (lactide-co-trimethylene carbonate) and poly (glycolide-co-trimethylene carbonate); (c) poly (ortho ester homopolymers and copolymers such as those synthesized by copolymerzation of various diketene acctals and diols; (d) polyanhydride homopolymers and copolymers such as poly (adipic anhydride), poly (suberic anhydride), poly (sebacic anhydride), poly (dodecanedioic anhydride), poly (maleic anhydride), poly[1,3-bis-(p-carboxyphenoxy) methane anhydride], and poly[alpha, omega-bis (p-carboxyphenoxy) alkane anhydride] such as poly[1,3-bis (p-carboxyphenoxy) propane anhydride] and poly[1,3-bis (p-carboxyphenoxy) hexane anhydride]; (c) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others.

A shape memory foam may have a thermal transition point (transition temperature) below which residual stress is maintained without a loading constraint. The thermal activation (which causes the shape memory) may be achieved with the desired material going through a semi-crystalline melt point or glass transition temperature between the first configuration and the expanded configuration. Several suitable thermal activation processes are known in the art and useful herein. In an example, the temperature activated memory shape foam may be formed for use as a medical device by first shaping a shape memory foam formed from a suitable material into its final expanded configuration; that is, the configuration that the shape memory shape foam will achieve once inserted into the left atrial appendage to provide the desired occlusive benefit. In this expanded configuration, the shape memory foam may generally have a diameter that will range from about 10 millimeters (about 0.39 inches) to about 50 millimeters (about 1.97 inches) and a length that will range from about 1 centimeter (about 0.39 inches) to about 5 centimeters (about 1.97 inches), although other diameters and lengths are within the scope of the present disclosure. Once this has been done, the foam may be heated above the transition temperature of the material; that is, the temperature at which a desired expansion will occur. Once the desired transition temperature has been achieved, the shape memory foam is held at a constant temperature and is re-shaped into an initial (unexpanded) configuration. This re-shaping is suitably done in a properly sized molding clement and may be any suitable shape. In this collapsed configuration, the shape memory foam will generally have a diameter of less than 4.7 millimeters (about 0.19 inches) and a length that will range from about 2 centimeters (about 0.79 inches) to about 5 centimeters (about 1.97 inches), although other diameters and lengths are within the scope of the present disclosure. After insertion into the molding element, the temperature is reduced to a temperature below the transition temperature to set the new shape; for example, the temperature may be reduced to room temperature to set the new shape. Once this has been completed, the shape memory foam will remain in its first configuration until it is subjected to a temperature at or above the transition temperature, at which time it will expand into its expanded, or remembered, configuration. In some instances, exposure to water within the blood changes the glass transition temperature of the foam. As an example, the shape memory foam may have a dry T_{g} (glass transition temperature) that is above body temperature, and may have a wet T_{g}, after exposure to water, that is lower than body temperature.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding the left atrial appendage (LAA), the LAAC device comprising a shape memory foam component having a compressed configuration in which the shape memory foam component has a first shape that is adapted for delivery and an expanded configuration in which the shape memory foam component has a second shape that different from the first shape and is adapted to occlude a left atrial appendage (LAA), the shape memory foam component formed by:
crimping a shape memory foam blank into an inverse shape that is an inverse of the second shape; and
subsequently cutting the shape memory foam component from the crimped shape memory foam blank, the shape memory foam component having the first shape.

2. The LAAC device of claim 1, wherein the second shape comprises a complex shape.

3. The LAAC device of claim 1, wherein the first shape comprises a cylindrical shape and the second shape comprises a non-cylindrical shape.

4. The LAAC device of claim 1, wherein the second shape is adapted to occlude an LAA having a profile that resembles a chicken wing.

5. The LAAC device of claim 1, wherein the second shape is adapted to occlude an LAA having a profile that resembles a windsock.

6. The LAAC device of claim 1, wherein the second shape is adapted to occlude an LAA having a profile that resembles a cactus.

7. The LAAC device of claim 1, wherein the second shape is adapted to occlude an LAA having a profile that resembles a cauliflower.

8. The LAAC device of claim 1, wherein the second shape is adapted to occlude an LAA having a double lobe.

9. The LAAC device of claim 1, wherein the shape memory foam component is adapted to change from the first shape to the second shape when exposed to increased temperature and/or increased moisture.

10. A method for manufacturing an occlusive device that includes a shape memory foam component having a compressed configuration in which the shape memory foam component has a first shape and an expanded configuration in which the shape memory foam component has a second shape different from the first shape, the method comprising:
crimping a shape memory foam blank into an inverse shape that is an inverse of the second shape;
heat setting the crimped shape memory foam blank to temporarily hold the crimped shape memory foam blank in the inverse shape; and
subsequently cutting the shape memory foam component from the crimped shape memory foam blank, the shape memory foam component having the first shape.

11. The method of claim 10, further comprising an initial step of cutting the shape memory foam blank out of a larger piece of shape memory foam prior to crimping the shape memory foam blank.

12. The method of claim 10, wherein the shape memory foam component is adapted to change from the first shape to the second shape when exposed to increased temperature and/or increased moisture.

13. The method of claim 10, wherein the first shape comprises a cylindrical shape.

14. The method of claim 10, wherein the second shape comprises a non-cylindrical shape.

15. The method of claim 10, wherein the second shape is adapted to occlude a left atrial appendage (LAA).

16. The method of claim 10, wherein the second shape is adapted to occlude a passage within a previously implanted device.

17. A method for manufacturing a left atrial appendage closure (LAAC) device that includes a shape memory foam component having a compressed configuration in which the shape memory foam component has a first shape that is adapted for delivery and an expanded configuration in which the shape memory foam component has a second shape that different from the first shape and is adapted to occlude a left atrial appendage (LAA), the method comprising:
placing a shape memory foam blank into a mold that is adapted to crimp the shape memory foam blank into an inverse shape that is an inverse of the expanded configuration;
using the mold to crimp the shape memory foam blank;
heat setting the crimped shape memory foam blank to temporarily hold the crimped shape memory foam blank in the inverse shape; and
cutting the shape memory foam component from the crimped shape memory foam blank, the shape memory foam component having the compressed configuration.

18. The method of claim 17, wherein the shape memory foam component is adapted to change from the first shape to the second shape when exposed to increased temperature and/or increased moisture.

19. The method of claim 17, wherein the first shape comprises a cylindrical shape.

20. The method of claim 17, wherein the second shape comprises a non-cylindrical shape.
